(19) United States
(12) Patent Application Publication
(10) Pub. No.: US 2009/0280491 A1
(43) Pub. Date: Nov. 12, 2009

(54) PREDICTING CANCER INVASIVENESS

(75) Inventors: Alain Borczuk, Roslyn Heights, NY (US); Brynn Levy, Closter, NJ (US); Charles A. Powell, River Vale, NJ (US)

Correspondence Address: SONNENSCHEIN NATH & ROSENTHAL LLP, P.O. BOX 061080, WACKER DRIVE STATION, WILLIS TOWER, CHICAGO, IL, 60606-1080, US

(73) Assignee: The Trustees of Columbia University in the City of New York, New York, NY (US)

(21) Appl. No.: 12/412,164

(22) Filed: Mar. 26, 2009

Related U.S. Application Data
(60) Provisional application No. 61/040,082, filed on Mar. 27, 2008.

(51) Int. Cl. C12Q 1/68

(52) U.S. Cl. 435/6

(57) ABSTRACT

Provided are methods of determining the likelihood of a human cancer being invasive. Also provided are methods of determining whether a lung adenocarcinoma is a bronchioloalveolar carcinoma (BAC). Additionally provided are methods of deciding a course of treatment for a patient with a cancer.

### CROSS-REFERENCE TO RELATED APPLICATIONS

This application claims priority to and the benefit of U.S. Provisional Application No. 61/040,082, filed Mar. 27, 2008, which is incorporated herein by reference in its entirety.

### BACKGROUND

Lung cancer metastasis represents the final step of a complex sequence comprised of invasion (loss of cell-cell adhesion, increased cell motility, and basement membrane degradation), vascular intravasation and extravasation, establishment of a metastatic niche, and angiogenesis (Fidler, 2003). Deciphering the molecular processes underlying the acquisition of invasiveness promises to have increasing importance as we anticipate a rise in the detection of early stage lung adenocarcinoma as a result of lung cancer screening with low-dose CT scans (Henschke et al., 1999; Swensen, 2002). Heterogeneity in clinical outcomes for patients with early stage lung adenocarcinoma is attributable in part to histological invasiveness.

The World Health Organization subclassifies adenocarcinoma based upon predominant cell morphology and growth pattern, such as bronchioloalveolar carcinoma (BAC), adenocarcinoma with mixed subtypes (AC-mixed), and homogenously invasive tumors with a variety of histological patterns (Brambilla et al., 2001). The histological distinction between BAC and other adenocarcinoma subclassifications is tissue invasion. BAC tumor cells are cuboidal to columnar, with or without mucin, which grow in a noninvasive fashion along alveolar walls. Invasion, defined as tumor disruption of the alveolar basement membrane, is present in other subtypes of adenocarcinoma. Adenocarcinoma with mixed subtypes frequently contains regions of noninvasive tumor at the periphery of invasive tumor. Tumor invasion results from autocrine and paracrine signaling events between and within the tumor epithelial cells and the stromal microenvironment (Bissel and Radisky, 2001; Elenbaas and Weinberg, 2001). Gene expression signatures of lung adenocarcinoma tumor specimens associated with invasion have been identified, along with repression of TGFBRII, as an important step in activating downstream Smad independent pathways to mediate invasion. Signaling events downstream of TGFBRII that are required for mediating invasion in TGFBRII repressed cells were also identified and characterized, such as the RANTES/CCR5 pathway, (Borczuk et al., 2005; 2008). A limitation of that genomics approach to identify tumor invasion signatures is that sections containing heterogeneous mixtures of tumor cells and stromal cells were utilized. This is adequate for the identification of global signatures but is inadequate for definitively distinguishing contributions of tumor cells from those of stromal cells. In a large-scale analysis of adenocarcinoma genomics, the contribution of stromal cells was estimated to range from 50-70% of tumor genomic signatures (Weir et al., 2007).

There is a need for improved methods and increased understanding of the biological properties of these tumors in order to discover diagnostic biomarkers and targeted therapeutics to enhance our treatment approaches for lung cancer and other cancers. The present invention addresses that need.

### SUMMARY

The inventors have identified an association between invasive human cancer and increased copy number and expression of genes in chromosome region 7q21, 7q22, 7q31 and/or 7q36. This association is useful for predicting the invasiveness of a cancer and assessing treatment options.

The invention is directed to methods of determining the likelihood of a human cancer being invasive. The methods comprise obtaining malignant cells of the cancer from a sample of tissue comprising the cancer, and comparing expression of a gene in chromosome region 7q21, 7q22, 7q31 or 7q36 in the malignant cells of the cancer with expression of the same gene in normal human cells. In these methods, increased expression of the gene in the malignant cells over the normal cells indicates the cancer is likely to be invasive, and expression of the gene in the malignant cells at or below the normal cells indicates the cancer is not likely to be invasive.

The invention is also directed to methods of determining whether a lung adenocarcinoma is a bronchioloalveolar carcinoma (BAC). The methods comprise obtaining malignant cells of the adenocarcinoma from a sample of tissue comprising the adenocarcinoma, and comparing expression of a gene in chromosome region 7q21, 7q22, 7q31 or 7q36 in the adenocarcinoma cells with expression of the same gene in normal human cells or in known BAC cells. In these methods, increased expression of the gene in the adenocarcinoma cells over normal or BAC cells indicates the adenocarcinoma is not a BAC, and expression of the gene in the adenocarcinoma cells at or below normal cells indicates the adenocarcinoma is a BAC.

Additionally, the invention is directed to methods of deciding a course of treatment for a patient with a cancer. The methods comprise obtaining malignant cells of the cancer from a sample of tissue comprising the cancer, and comparing expression of a gene in chromosome region 7q21, 7q22, 7q31 or 7q36 in the malignant cells of the cancer with expression of the same gene in normal human cells. In these methods, increased expression of the gene in the malignant cells over normal cells indicates the patient should undergo an aggressive course of treatment, and expression of the gene in the malignant cells at or below normal cells indicates the patient should not undergo an aggressive course of treatment.

### BRIEF DESCRIPTION OF THE DRAWINGS

FIG. 1 is a hierarchical dendrogram tree of the 40 cases in the Example 1 study of microdissected BAC and mixed subtype adenocarcinoma. This tree is reproducible and the expectation is that multiple analyses will yield the same general tree. The tree demonstrates 2 main classes that on the left have more of the invasive tumors while the tree on the right shows more of the BAC/in situ tumors.

FIG. 2 is a graph of the two classes. When the two classes are compared in situ/non-invasive versus mixed invasive a set of genes is determined which is associated with each class. The distribution of the genes on that list showed a preponderance of genes on chromosome 7 as indicated by the right bar.

FIG. 3 shows the results of the expression of 109 probe sets that were differentially expressed on 7q, where the probes are darkly shaded if their expression is above the mean and lightly shaded if below mean expression. White cells indicate the probe set was approximately at the mean. This is done by case, each column is one case, with the BAC cases on the left and the mixed cases on the right. The relatively high expression in the mixed cases, as demonstrated by dark boxes shows the finding was present for many of the cases although not all the genes were increased in all of the cases.

FIG. 4 is a graph that maps all the genes on 7q in order of occurrence, where each band represents a single gene. The genes with increased expression on the more stringent statistical list described in Example 1 are darkly shaded while the ones on the longer list with lower stringency are lightly shaded if increased in mixed subtype tumors. This demonstrates that the genes increased in mixed subtype tumors are not randomly distributed over the chromosome, but in fact are in clusters. This suggests that there are DNA structural changes that explain the increased expression, and the regions mapped by this includes regions 7q21, 7q22, 7q31 and 7q36.

FIG. 5 is a diagram outlining the whole genome amplification technique. This technique linearly amplifies DNA so that sufficient quantities are available for DNA based studies.

FIG. 6 is a chromosomal ideogram shows the relative DNA quantities between pooled mixed subtype tumor and pooled BAC as performed by conventional comparative genomic hybridization (CGH). The bars on the right next to the chromosome represent regions of DNA increase in mixed (or decrease in BAC), and bars on the left represent regions increased in BAC or decreased in mixed. Since this is a relative test between 2 tumor type, we can say that 7q is relatively higher in mixed than BAC, and for example 8q is relatively lower in mixed than BAC.

FIG. 7 is a diagram showing comparative genomic hybridization (CGH) analysis using individual BAC and Mixed tumors vs. normal diploid DNA. These studies confirmed 7q deletion in a subset of BAC tumors and focal chromosomal amplifications in mixed tumors, as well as a uniform amplification of the 7p EGFR locus in BAC and in most mixed tumors.

### DETAILED DESCRIPTION

The inventors have discovered that invasive human cancers are associated with an increase in copy number of genes in chromosome regions 7q21, 7q22, 7q31 and 7q36. The increased copy number is reflected in an increased expression of genes in those regions. Thus, the increase in copy number can be detected by measuring expression of the genes.

As used herein, a cancer is invasive if it has the ability to disrupt and spread beyond a basement membrane. Invasive cancers generally carry a poorer prognosis than non-invasive cancers, since invasive cancers are not delimited by basement membrane barriers and can metastasize to other areas of the body. Being able to predict whether a cancer is invasive allows the oncologist to accurately formulate an appropriate treatment regimen based on the cancer's likelihood of spreading and having a poor prognosis. Thus, an invasive cancer would generally be treated more aggressively than a cancer that will not spread.

The invention is directed to methods of determining the likelihood of a human cancer being invasive. The methods comprise obtaining malignant cells of the cancer from a sample of tissue comprising the cancer, and comparing expression of a gene in chromosome region 7q21, 7q22, 7q31 or 7q36 in the malignant cells of the cancer with expression of the same gene in normal human cells. In these methods, increased expression of the gene in the malignant cells over the normal cells indicates the cancer is likely to be invasive, and expression of the gene in the malignant cells at or below the normal cells indicates the cancer is not likely to be invasive.

In some embodiments, the gene analyzed in these methods is within the chromosome 7q nucleotide range 97629065-97744861, 988836407-99069750, 99350773-99883433, 100942737-101517029, 104421612-104557622, 106027489-106136511, 111410241-111526144, 130221442-130346785, 138356096-138465713, 139348161-139662180, 148682566-148839629, 149045357-149210881, 150011920-151485535, 155032354-155171735, or 156713827-158812469.

In other embodiments, the gene is within chromosome 7q nucleotide range 100995284-101955975, 157831237-158160305, or 158167149-158726832. Nonlimiting examples of genes in those regions are EMID2, MYLC2PL, CUX1, SH2B2, PRKRIP1, ALKBH4, LRWD1, POLR2J, ORAI2, PTPRN2, WDR60, VIPR2, FAM62B or NCAPG2. As established in Example 2, at least CUX1 and PTPRN2 have increased expression.

For these methods, the expression of the gene in the malignant cells may be determined substantially separately from stromal cells that were associated with the malignant cells in vivo, as in the examples below. Thus, the malignant cells can advantageously be substantially separated from stromal cells. This separation can be executed by any known method, for example expression microdissection or, as in Example 1, laser capture micro dissection.

Without being bound to any particular mechanism, it is believed that regions 7q21, 7q22, 7q31 and 7q36 comprise a gene or genes that contributes to cancer invasiveness, either directly or by signal transduction.

Thus, these methods are expected to be useful for determining invasiveness of any cancer, including but not limited to solid tumors, cutaneous tumors, melanoma, malignant melanoma, renal cell carcinoma, colorectal carcinoma, colon cancer, lymphomas (including glandular lymphoma), Kaposi's sarcoma, prostate cancer, kidney cancer, ovarian cancer, lung cancer, head and neck cancer, pancreatic cancer, mesenteric cancer, gastric cancer, rectal cancer, stomach cancer, bladder cancer, leukemia (including hairy cell leukemia and chronic myelogenous leukemia), breast cancer, non-melanoma skin cancer (including squamous cell carcinoma and basal cell carcinoma), and glioma. In certain embodiments, the cancer is a lung cancer, e.g., an epithelial neoplasm, such as a papilloma, a carcinoma, an adenocarcinoma, a ductal lobular or medullary carcinoma, an acinic cell carcinoma, a complex epithelial carcinoma, a gonadal tumor, a paragangioma, a glomus tumor, or a melanoma. In particular embodiments, the cancer is an adenocarcinoma, e.g., a lung adenocarcinoma, including but not limited to an insulinoma, a glucagonoma, a gastrinoma, VIPoma, a somatostatinoma, or a cholangiocarcinoma.

The invention methods can further comprise comparing the expression of a second gene in the malignant cells with expression of the second gene in normal cells. The second gene can be any gene associated with cancer invasiveness such as HOXC10, CCL₅ (RANTES), or CCR₅ (positively associated with invasiveness, see Zhai et al., 2007 and Borczuk et al., 2008) or TGFBRII (negatively associated with invasiveness, see Dong et al., 2007). In other embodiments, the second gene is in chromosome region 7q21, 7q22, 7q31 or 7q36. The expression of any number of additional genes, e.g., associated with cancer invasiveness or any other trait, may also be evaluated as part of these methods.

In these methods, expression of the gene in the malignant cells can be determined by any known method. For example, the product of the gene can be quantified with antibodies or by any other method. In other embodiments, expression of the gene in the malignant cells is determined by quantifying mRNA of the gene, e.g., by PCR methods (for example RT-PCR). In additional embodiments, expression of the gene in the malignant cells is determined by determining copy number of the gene. Here, a copy number higher than 2 generally indicates increased expression of the gene and a copy number of 2 or lower generally indicates no increased expression of the gene. Copy number can be determined by any known method, for example comparative genomic hybridization methods, e.g., using fluorescence in situ hybridization (FISH) or real-time PCR. Copy number of a gene can also be determined by determining the copy number of a chromosomal region adjacent to, or near the gene.

The invention is also directed to methods of determining whether a lung adenocarcinoma is a bronchioloalveolar carcinoma (BAC). The methods comprise obtaining malignant cells of the adenocarcinoma from a sample of tissue comprising the adenocarcinoma, and comparing expression of a gene in chromosome region 7q21, 7q22, 7q31 or 7q36 in the adenocarcinoma cells with expression of the same gene in normal human cells or in known BAC cells. In these methods, increased expression of the gene in the adenocarcinoma cells over normal or BAC cells indicates the adenocarcinoma is not a BAC, and expression of the gene in the adenocarcinoma cells at or below normal cells indicates the adenocarcinoma is a BAC.

In some embodiments, the gene analyzed in these methods is within the chromosome 7q nucleotide range 97629065-97744861, 988836407-99069750, 99350773-99883433, 100942737-101517029, 104421612-104557622, 106027489-106136511, 111410241-111526144, 130221442-130346785, 138356096-138465713, 139348161-139662180, 148682566-148839629, 149045357-149210881, 150011920-151485535, 155032354-155171735, or 156713827-158812469.

In other embodiments, the gene is within chromosome 7q nucleotide range 100995284-101955975, 157831237-158160305, or 158167149-158726832. Nonlimiting examples of genes in those regions are EMID2, MYLC2PL, CUX1, SH2B2, PRKRIP1, ALKBH4, LRWD1, POLR2J, ORAI2, PTPRN2, WDR60, VIPR2, FAM62B or NCAPG2, in particular CUX1 and PTPRN2.

For these methods, the expression of the gene in the malignant cells of the adenocarcinoma can be determined substantially separately from stromal cells that were associated with the malignant cells in vivo. In those embodiments, the malignant cells are substantially separated from stromal cells. This separation can be executed by any known method, for example expression microdissection or laser capture microdissection.

These invention methods can also further comprise comparing the expression of a second gene in the malignant cells with expression of the second gene in normal cells. The second gene can be any gene associated with cancer invasiveness such as HOXC10, CCL₅ (RANTES), CCR₅ or TGFBRII. In other embodiments the second gene is in chromosome region 7q21, 7q22, 7q31 or 7q36. The expression of any number of additional genes, e.g., associated with cancer invasiveness or any other trait, may also be evaluated as part of these methods.

As in the methods described above, in these methods expression of the gene in the malignant cells can be determined by any known method. For example, the product of the gene can be quantified with antibodies or by any other method. Expression of the gene in the malignant cells of the adenocarcinoma can also be determined by quantifying mRNA of the gene, e.g., by PCR methods (for example RT-PCR). In other embodiments, expression of the gene in the malignant cells is determined by determining copy number of the gene. Here, a copy number higher than 2 generally indicates increased expression of the gene and a copy number of 2 or lower generally indicates no increased expression of the gene. Copy number can be determined by any known method, for example comparative genomic hybridization methods, e.g., using fluorescence in situ hybridization (FISH) or real-time PCR. Copy number of a gene can also be determined by determining the copy number of a chromosomal region adjacent to, or near the gene.

Additionally, the invention is directed to methods of deciding a course of treatment for a patient with a cancer. The methods comprise obtaining malignant cells of the cancer from a sample of tissue comprising the cancer, and comparing expression of a gene in chromosome region 7q21, 7q22, 7q31 or 7q36 in the malignant cells of the cancer with expression of the same gene in normal human cells. In these methods, increased expression of the gene in the malignant cells over normal cells indicates the patient should undergo an aggressive course of treatment, and expression of the gene in the malignant cells at or below normal cells indicates the patient should not undergo an aggressive course of treatment.

In some embodiments, the gene analyzed in these methods is within the chromosome 7q nucleotide range 97629065-97744861, 988836407-99069750, 99350773-99883433, 100942737-101517029, 104421612-104557622, 106027489-106136511, 111410241-111526144, 130221442-130346785, 138356096-138465713, 139348161-139662180, 148682566-148839629, 149045357-149210881, 150011920-151485535, 155032354-155171735, or 156713827-158812469.

In other embodiments, the gene is within chromosome 7q nucleotide range 100995284-101955975, 157831237-158160305, or 158167149-158726832. Nonlimiting examples of genes in those regions are EMID2, MYLC2PL, CUX1, SH2B2, PRKRIP1, ALKBH4, LRWD1J, POLR2J, ORAI2, PTPRN2, WDR60, VIPR2, FAM62B or NCAPG2, in particular CUX1 and PTPRN2.

For these methods, the expression of the gene in the malignant cells can be determined substantially separately from stromal cells that were associated with the malignant cells in vivo. In those embodiments, the malignant cells are substantially separated from stromal cells. This separation can be executed by any known method, for example expression microdissection or laser capture microdissection.

These invention methods can also further comprise comparing the expression of a second gene in the malignant cells with expression of the second gene in normal cells. The second gene can be any gene associated with cancer invasiveness such as HOXC10, CCL₅ (RANTES), CCR₅ or TGFBRII. In other embodiments the second gene is in chromosome region 7q21, 7q22, 7q31 or 7q36. The expression of any number of additional genes, e.g., associated with cancer invasiveness or any other trait, may also be evaluated as part of these methods.

As in the methods described above, in these methods expression of the gene in the malignant cells can be determined by any known method. For example, the product of the gene can be quantified with antibodies or by any other method. Expression of the gene in the malignant cells can also be determined by quantifying mRNA of the gene, e.g., by PCR methods (for example RT-PCR). In other embodiments, expression of the gene in the malignant cells is determined by determining copy number of the gene. Here, a copy number higher than 2 generally indicates increased expression of the gene and a copy number of 2 or lower generally indicates no increased expression of the gene. Copy number can be determined by any known method, for example comparative genomic hybridization methods, e.g., using fluorescence in situ hybridization (FISH) or real-time PCR. Copy number of a gene can also be determined by determining the copy number of a chromosomal region adjacent to, or near the gene.

These methods are useful for deciding a course of treatment for any cancer, including but not limited to solid tumors, cutaneous tumors, melanoma, malignant melanoma, renal cell carcinoma, colorectal carcinoma, colon cancer, lymphomas (including glandular lymphoma), Kaposi's sarcoma, prostate cancer, kidney cancer, ovarian cancer, lung cancer, head and neck cancer, pancreatic cancer, mesenteric cancer, gastric cancer, rectal cancer, stomach cancer, bladder cancer, leukemia (including hairy cell leukemia and chronic myelogenous leukemia), breast cancer, non-melanoma skin cancer (including squamous cell carcinoma and basal cell carcinoma), and glioma. In certain embodiments, the cancer is a lung cancer, e.g., an epithelial neoplasm, such as a papilloma, a carcinoma, an adenocarcinoma, a ductal lobular or medullary carcinoma, an acinic cell carcinoma, a complex epithelial carcinoma, a gonadal tumor, a paragangioma, a glomus tumor, or a melanoma. In particular embodiments, the cancer is an adenocarcinoma, e.g., a lung adenocarcinoma, including but not limited to an insulinoma, a glucagonoma, a gastrinoma, VIPoma, a somatostatinoma, or a cholangiocarcinoma.

Preferred embodiments are described in the following examples. Other embodiments within the scope of the claims herein will be apparent to one skilled in the art from consideration of the specification or practice of the invention as disclosed herein. It is intended that the specification, together with the example, be considered exemplary only, with the scope and spirit of the invention being indicated by the claims, which follow the examples.

### Example 1

### Gene Signatures of Invasiveness in Adenocarcinoma

To examine gene profiles associated with adenocarcinoma (AdCa) heterogeneity and invasiveness and to understand matrix/epithelial cell interactions that mediate this process, laser capture microdissection methods were utilized. Using these methods, tumor cells from BAC and AC-mixed tumors were analyzed separately.

Tumor cells from frozen sections of 17 BAC and 23 mixed subtype AdCa were dissected using the PALM Microbeam laser capture microscope (LCM). RNA quality after microdissection was evaluated with the Agilent 2100 Bioanalyzer and processed for hybridization to Affymetrix U133 Plus 2.0 arrays using standard protocols (Borczuk et al., 2004) and data were normalized using GCRMA. All samples passed quality control metrics. These metrics included Distribution of Affymetrix MAS 5.0 average background; distribution of scaling factors; percent genes present; Actin and GAPDH ratios; and output of RND degradation, RLE and NUSE plots (Bolstad et al., 2005). To verify the precision of the microdissection, mRNA expression values were examined for cell lineage specific genes representative of epithelial vs. non-epithelial cells. It was determined that the specimens were enriched for epithelial associated genes.

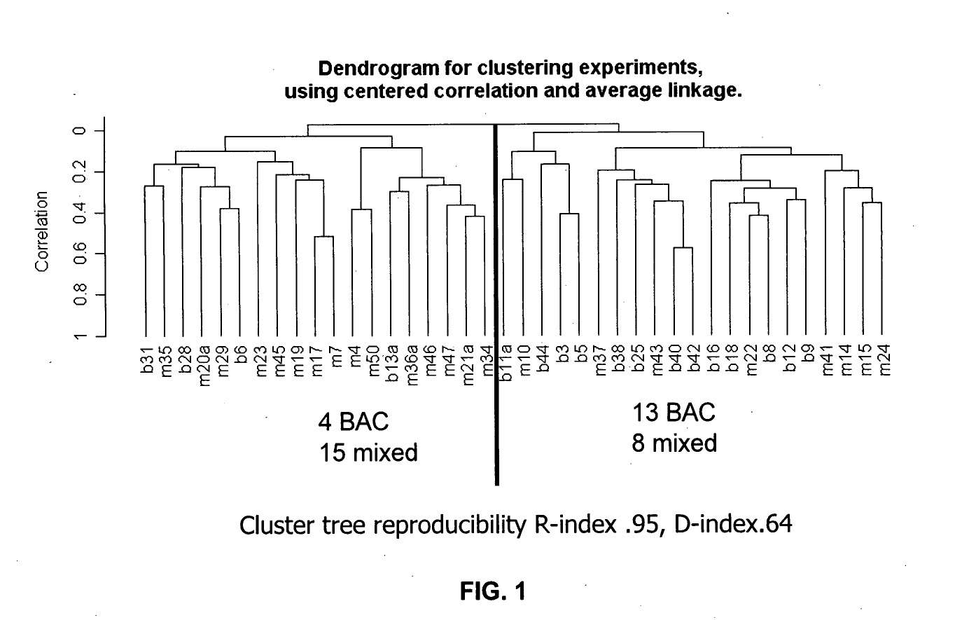

Unsupervised hierarchical clustering identified two reproducible main clusters. Fifteen of 23 mixed subtype AdCa were located in cluster 1, and 13 of 17 BAC were located in cluster 2 (Fisher p=0.01) indicating a distinct global gene expression between BAC and AC-mixed (FIG. 1). To determine if clustering was related to activation of pathways downstream of KRAS and EGFR, tumor DNA was examined for the prevalence of mutations in all tumor specimens. EGFR mutations were frequent and more common in the BAC cluster, as expected. KRAS mutations were relatively infrequent (˜10%) in BAC and AC-mixed tumors. Taken together, these results suggest that BAC and AC-Mixed signatures derived from LCM captured tumor cells are distinct and are independent of EGFR and KRAS mutation status.

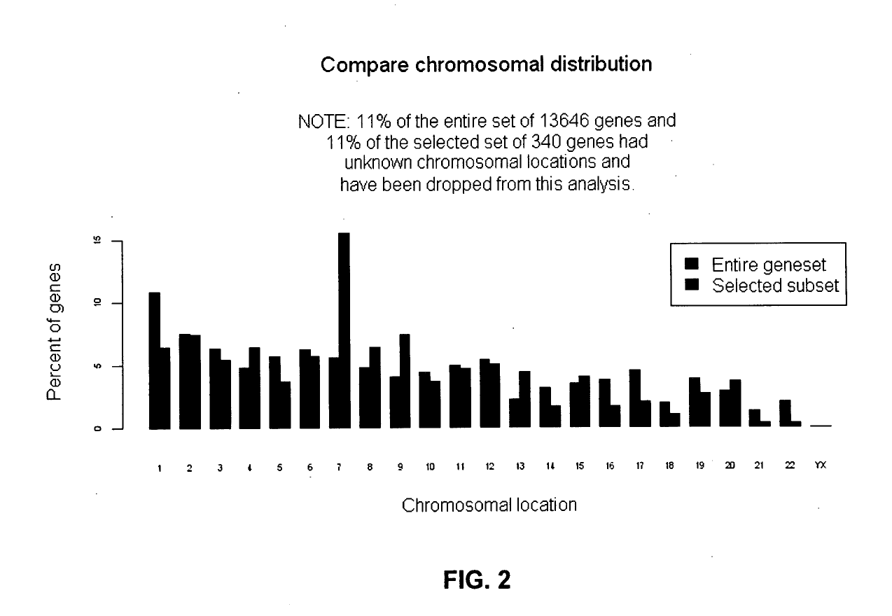

Supervised analysis was performed using an F-test within BRB array tools (Simon et al., 2007) to identify genes associated with histological subtype. 340 genes were differentially expressed between the two subclasses (P<0.01). The chromosomal distribution of the 340 gene signature was examined. Significant overrepresentation of genes from chromosomes 7, 8, 9, 13 was identified, with the greatest percentage of differentially expressed genes located on chromosome 7 (FIG. 2).

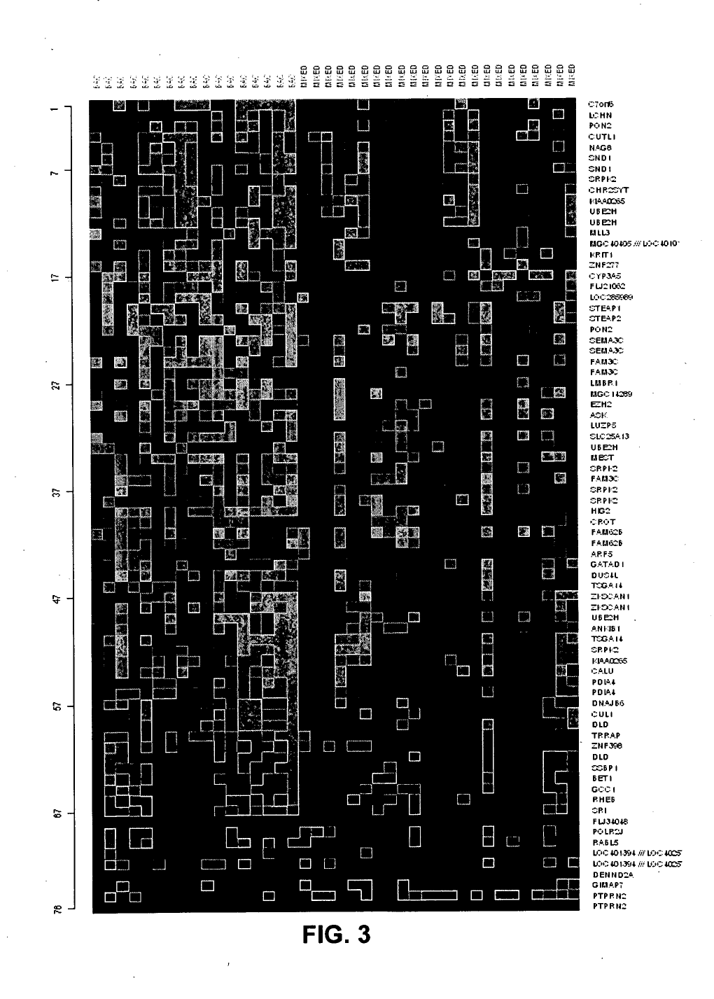

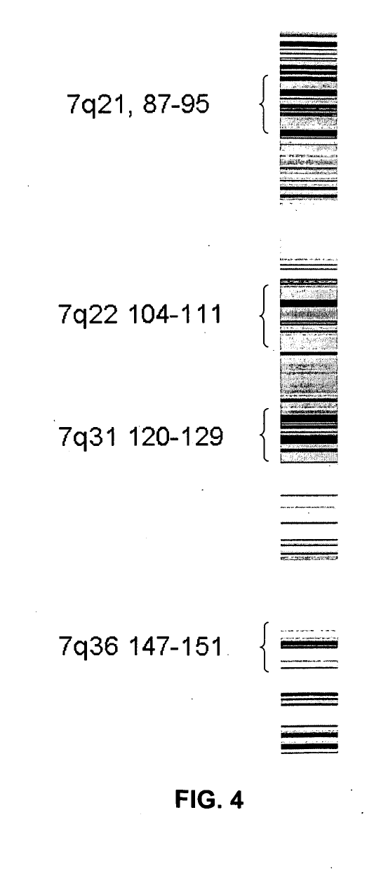

Expression of chromosome 7 genes was consistently higher in the invasive AC-mixed subtype specimens. The 340 gene invasion signature contained 66 probe sets representing 31 genes from 7q. Fifty-seven probe sets from 28 genes were localized to 7q21, 7q22, 7q31, and 7q36 and showed increased expression in mixed subtype by 1.5 fold or greater. To determine if this result was generalizeable beyond the selected set of genes included in the F-test signature, normalized mRNA expression values were examined for all chromosome 7q genes represented on the Hu133 Plus 2.0 microarray (Affymetrix) (FIG. 3). Those results show clusters of overexpressed genes in invasive tumors that are localized to specific loci of chromosome 7q and are suggestive of focal chromosomal amplification (FIG. 4). Taken together, the microarray mRNA expression data suggest that the gene expression increases in Mixed subtype AdCa may be related to structural copy number increases (i.e. amplification) in chromosome 7q.

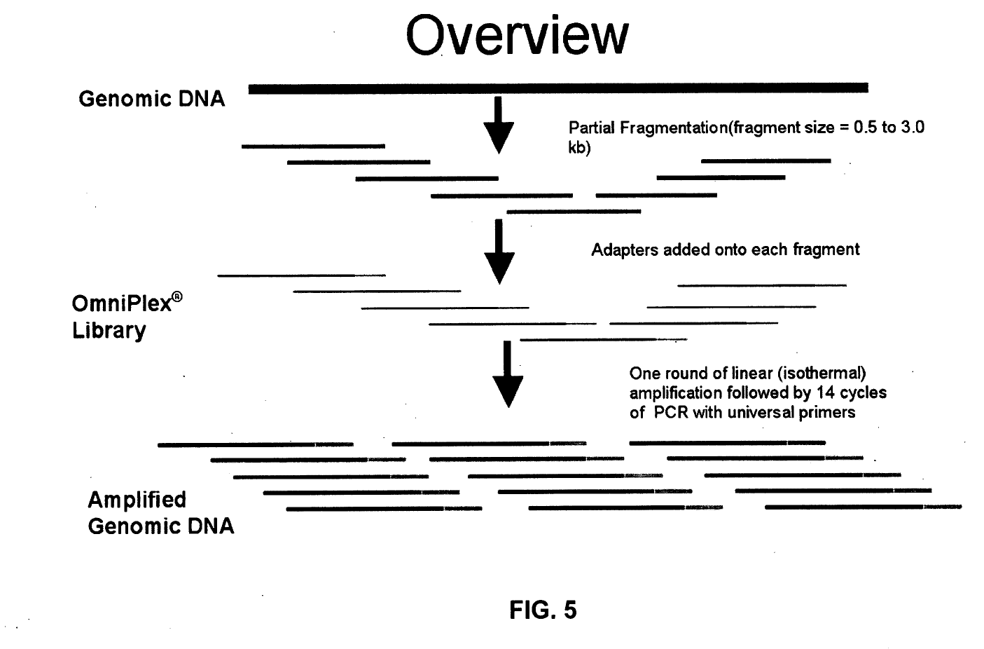

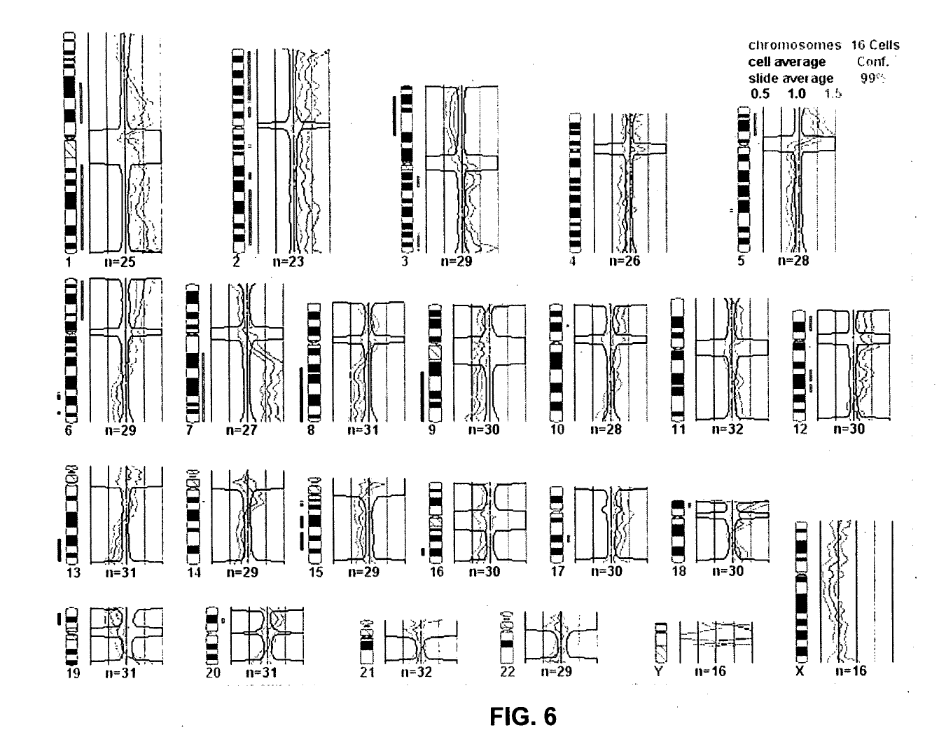

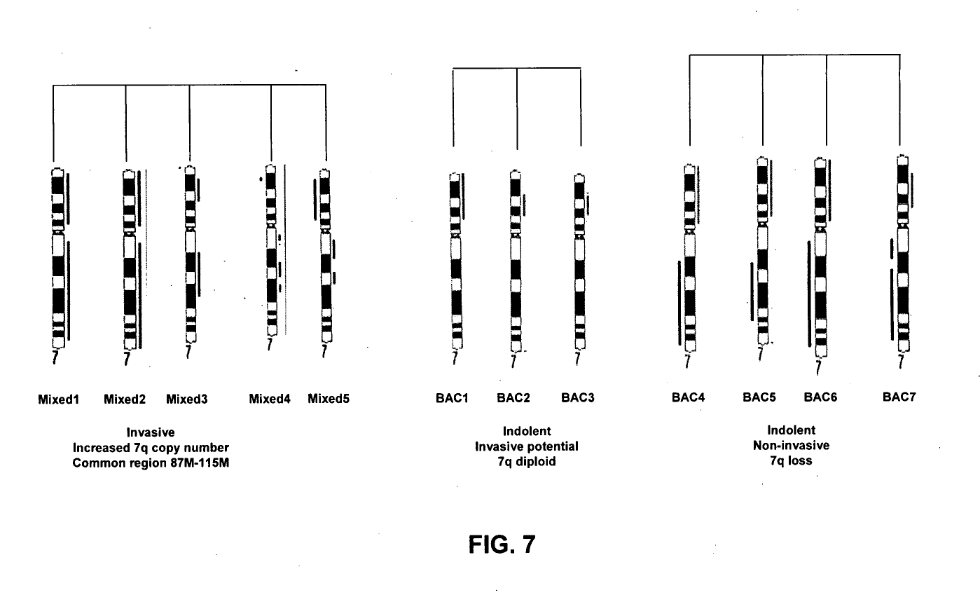

To examine structural copy number changes, comparative genomic hybridization (CGH) analysis was performed on metaphase spreads using whole-genome amplified DNA (Brueck et al., 2007) (FIGS. 5 and 6). The CGH profiles were compared to a dynamic reference standard based upon an average of normal cases. In each case approximately 15 cells were counted: chromosome regions where the 99% confidence interval included 1.5 fold copy changes were considered positive. CGH of 9 pooled Mixed vs. BAC tumors showed 1.5 fold copy number increase in chromosome 7q and of 9 pooled Mixed vs. normal diploid DNA showed increase in 7q11, 7q21-22, 7q31-32, and 7q35-36 as well as in 7p at the EGFR locus. These results were confirmed using genomic qRT-PCR for representative chromosome 7q genes TRRAP (Transformation/transcription domain-associated protein, 7q21.2) and FAM3C (Family with sequence similarity 3, member C, 7q31). Using the PRISM 7500 sequence detection kit and inventory TaqMan primers, the standard curve method was used to calculate gene copy number in tumor DNA sample relative to a reference, the RNAse P gene. The correlation between copy number and gene expression (Spearman rank coefficient) was 0.352 (p<0.03) for TRRAP and 0.667 (p<0.003) and 0.529 (p<0.02) for the two probe sets representing FAM3C. Importantly, a reduction of copy number was detected in a subset of BAC tumors relative to reference diploid DNA. To confirm this observation, additional CGH analysis was performed using individual BAC and Mixed tumors vs. normal diploid DNA (FIG. 7). These studies confirmed 7q deletion in a subset of BAC tumors and they confirmed focal chromosomal amplifications in Mixed tumors as well as a uniform amplification of the 7p EGFR locus in BAC and in most Mixed tumors. These findings suggest the following paradigm: 1. As shown by others, EGFR alterations drive proliferation in these tumor subtypes; 2. Amplification of 7q loci promotes invasion in adenocarcinoma; 3. Deletion of 7q loci in BAC tumors may prevent the acquisition of invasion.

Taken together, these experiments indicate lung adenocarcinoma invasive cases are associated with increased expression of 7q genes, with a mechanism related to increased copy number. The 7q regions most associated with this increased expression are within the chromosome 7q nucleotide range 97629065-97744861, 988836407-99069750, 99350773-99883433, 100942737-101517029, 104421612-104557622, 106027489-106136511, 111410241-111526144, 130221442-130346785, 138356096-138465713, 139348161-139662180, 148682566-148839629, 149045357-149210881, 150011920-151485535, 155032354-155171735, or 156713827-158812469. The distribution of the regions of interest suggest focal chromosomal amplification rather than polysomy as the mechanism of copy number increase and they identify regions distinct from those harboring genes known to be important for lung adenocarcinoma pathogenesis (EGFR-7p, MET 7q31, and BRAF 7q34 [Engelman et al., 2007; Paez et al., 2004; Shigematsu and Gazdar, 2006]).

### Example 2

### Further Characterization of Chromosomal Regions Associated with Adenocarcinoma Invasiveness

To further define the region of amplification, non-amplified DNA was obtained from frozen tissue specimens of invasive adenocarcinoma by laser capture microdissection to obtain sufficient material for high density oligonucleotide single nucleotide polymorphism arrays (Affymetrix Genome-Wide Human SNP Array 6.0). These arrays provide information on 946,000 probes for copy number variation. Using these results and subsequent analysis of overlapping consensus regions, two regions of interest were discovered, Region 1 and Region 2, as described below.

Region 1—Table 1 shows genes in this consensus region.

TABLE 1

| Gene | | | | Gene | Length |
|---|---|---|---|---|---|
| Symbol | Chrom.^{a} | Start | End | overlap^{b} | (bp) |
| MYLC2PL | 7 | 100995284 | 101955975 | 1 | 960692 |
| CUX1 | 7 | 100995284 | 101955975 | 1 | 960692 |
| SH2B2 | 7 | 100995284 | 101955975 | 1 | 960692 |
| PRKRIP1 | 7 | 100995284 | 101955975 | 1 | 960692 |
| ALKBH4 | 7 | 100995284 | 101955975 | 1 | 960692 |
| LRWD1 | 7 | 100995284 | 101955975 | 1 | 960692 |
| POLR2J | 7 | 100995284 | 101955975 | 1 | 960692 |
| ORAI2 | 7 | 100995284 | 101955975 | 1 | 960692 |

^{a}Chromosome number;
^{b}Proportion of overlap, where 1 = 100%

Based on the gene expression data, CUX1 is the gene whose expression is increased based on this region of increased copy number. However, the amplicon is a 960692 base pair region that contains 7 other genes, all of which show a copy number increase and could be used in a test of increased copy number such as FISH or real-time PCR for copy number analysis.

Region 2—Table 2 shows genes in this consensus region.

| Gene | | | | Gene | Length |
|---|---|---|---|---|---|
| Symbol | Chrom.^{a} | Start | End | overlap^{b} | (bp) |
| PTPRN2 | 7 | 157831237 | 158160305 | 0.230715 | 329069 |
| WDR60 | 7 | 158167149 | 158726832 | 1 | 559684 |
| VIPR2 | 7 | 158167149 | 158726832 | 1 | 559684 |
| FAM62B | 7 | 158167149 | 158726832 | 1 | 559684 |
| NCAPG2 | 7 | 158167149 | 158726832 | 0.314644 | 559684 |

^{a}Chromosome number;
^{b}Proportion of overlap, where 1 = 100%

Based on the gene expression data, PTPRN2 is the gene whose expression is increased based on this region of increased copy number. This 329069 base pair region is contiguous to a region of 559684 containing 4 additional genes, whose copy number could be used for a test of increased copy number such as FISH or real-time PCR for copy number analysis.

### REFERENCES

Bissell M J, Radisky D. 2001. Putting tumours in context. Nat Rev Cancer; 1:46-54.
Bolstad B M, Collin F, Brettscneider J, Simpson K, Cope L M, Irizarry R, Speed T P. Quality Assessment of Affymetrix GeneChip Data. In: Gentelman R, Carey V, Huber W, Dudoit S, eds. Bioinformatics and Computational Biology Solutions using R and Bioconductor. New York: Springer; 2005.
Borczuk A C, Shah L, Pearson G D N, et al. 2004. Molecular Signatures in Biopsy Specimens of Lung Cancer. Am J Respir Crit Care Med; 170:167-74.
Borczuk A C, Kim H K, Yegen H A, Friedman R A, Powell C A. 2005. Lung adenocarcinoma global profiling identifies type II transforming growth factor-beta receptor as a repressor of invasiveness. Am J Respir Crit Care Med; 172:729-37.
Borczuk A C, Papanikolaou N, Toonkel R L, et al. 2008. Lung adenocarcinoma invasion in TGFβ RII-deficient cells is mediated by CCL5/RANTES. Oncogene; 27:557-64.
Borczuk A C, Cappellini G C, Kim H K, Hesdorffer M, Taub R N, Powell C A. 2007. Molecular profiling of malignant peritoneal mesothelioma identifies the ubiquitin-proteasome pathway as a therapeutic target in poor prognosis tumors. Oncogene; 26:610-7.
Brambilla E, Travis W D, Colby T V, Corrin B, Shimosato Y. 2001. The new World Health Organization classification of lung tumours. Eur Respir J; 18:1059-68.
Brueck C, Song S, Collins J. 2007. Oligonucleotide Array CGH Analysis of a Robust Whole Genome Amplification Method. Biotechniques; 42:230-3.
Dong M, et al. 2007. The type III TGF-β receptor suppresses breast cancer progression. J Clin Invest; 117:206-17.
Elenbaas B, Weinberg R A. 2001. Heterotypic signaling between epithelial tumor cells and fibroblasts in carcinoma formation. Exp Cell Res; 264:169-84.
Engelman J A, Zejnullahu K, Mitsudomi T, et al. 2007. MET amplification leads to gefitinib resistance in lung cancer by activating ERBB3 signaling. Science (New York, N.Y.; 316:1039-43.
Fidler I J. 2003. The pathogenesis of cancer metastasis: the ‘seed and soil’ hypothesis revisited. Nat Rev Cancer; 3:453-8.
Futreal P A, Coin L, Marshall M, et al. 2004. A census of human cancer genes. Nat Rev Cancer; 4:177-83.
Henschke C I, McCauley D I, Yankelevitz D F, et al. 1999. Early Lung Cancer Action Project: overall design and findings from baseline screening. Lancet; 354:99-105.
Kim H, Xu G L, Borczuk A C, et al. 2003. The heparan sulfate proteoglycan GPC3 is a potential lung tumor suppressor. Am J Respir Cell Mol Biol; 29:694-701
Paez J G, Janne P A, Lee J C, et al. 2004. EGFR Mutations in Lung Cancer: Correlation with Clinical Response to Gefitinib Therapy. Science; 304:1497-500.
Shigematsu H, Gazdar A F. 2006. Somatic mutations of epidermal growth factor receptor signaling pathway in lung cancers. International journal of cancer; 118:257-62.
Siebert R, Jacobi C, Matthiesen P, et al. 1998. Detection of deletions in the short arm of chromosome 3 in uncultured renal cell carcinomas by interphase cytogenetics. The Journal of urology; 160:534-9.
Simon R, Radmacher R, Bittner M. BRB Tools. In. 3.5 ed: National Cancer Institute; 2007.
Swensen S J, Jett J R, Sloan J A, et al. 2002. Screening for lung cancer with low-dose spiral computed tomography. Am J Respir Crit Care Med; 165:508-13.
Weir B A, Woo M S, Getz G, et al. 2007. Characterizing the cancer genome in lung adenocarcinoma. Nature; 450:893-8.
Zhai Y et al. 2007. Gene expression analysis of preinvasive and invasive cervical squamous cell carcinomas identifies HOXC10 as a key mediator of invasion. Cancer Res.; 67:10163-10172.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

What is claimed is:

1. A method of determining the likelihood of a human cancer being invasive, the method comprising
obtaining malignant cells of the cancer from a sample of tissue comprising the cancer, and
comparing expression of a gene in chromosome region 7q21, 7q22, 7q31 or 7q36 in the malignant cells of the cancer with expression of the same gene in normal human cells,
wherein increased expression of the gene in the malignant cells over the normal cells indicates the cancer is likely to be invasive, and expression of the gene in the malignant cells at or below the normal cells indicates the cancer is not likely to be invasive.

2. The method of claim 1, wherein the gene is within chromosome 7q nucleotide range 97629065-97744861, 988836407-99069750, 99350773-99883433, 100942737-101517029, 104421612-104557622, 106027489-106136511, 111410241-111526144, 130221442-130346785, 138356096-138465713, 139348161-139662180, 148682566-148839629, 149045357-149210881, 150011920-151485535, 155032354-155171735, or 156713827-158812469.

3. The method of claim 2, wherein the gene is within chromosome 7q nucleotide range 100995284-101955975, 157831237-158160305, or 158167149-158726832.

4. The method of claim 3, wherein the gene is EMID2, MYLC2PL, CUX1, SH2B2, PRKRIP1, ALKBH4, LRWD1, POLR2J, ORAI2, PTPRN2, WDR60, VIPR2, FAM62B or NCAPG2.

5. The method of claim 3, wherein the gene is CUX1 or PTPRN2.

6. The method of claim 1, wherein the expression of the gene in the malignant cells is determined substantially separately from stromal cells that were associated with the malignant cells in vivo.

7. The method of claim 6, wherein the malignant cells are substantially separated from stromal cells by laser capture microdissection.

8. The method of claim 1, wherein the cancer is an adenocarcinoma.

9. The method of claim 1, wherein the cancer is a lung cancer.

10. The method of claim 1, wherein the cancer is a lung adenocarcinoma.

11. The method of claim 1, wherein expression of a second gene in the malignant cells is compared with expression of the second gene in normal cells.

12. The method of claim 11, wherein the second gene is in chromosome region 7q21, 7q22, 7q31 or 7q36.

13. The method of claim 11, wherein the second gene is HOXC10, CCL₅ (RANTES), CCR₅ or TGFBRII.

14. The method of claim 1, wherein expression of the gene in the malignant cells is determined by quantifying mRNA of the gene.

15. The method of claim 14, wherein mRNA of the gene is quantified using PCR.

16. The method of claim 1, wherein expression of the gene in the malignant cells is determined by determining copy number of the gene, wherein a copy number higher than 2 indicates increased expression of the gene and a copy number of 2 or less indicates expression of the gene at or below normal cells.

17. The method of claim 16, wherein copy number of a series of contiguous genes is determined.

18. The method of claim 17, wherein copy number determination is made by comparative genomic hybridization analysis.

19. A method of determining whether a lung adenocarcinoma is a bronchioloalveolar carcinoma (BAC), the method comprising
obtaining malignant cells of the adenocarcinoma from a sample of tissue comprising the adenocarcinoma, and
comparing expression of a gene in chromosome region 7q21, 7q22, 7q31 or 7q36 in the adenocarcinoma cells with expression of the same gene in normal human cells or in known BAC cells,
wherein increased expression of the gene in the adenocarcinoma cells over normal or BAC cells indicates the adenocarcinoma is not a BAC, and expression of the gene in the adenocarcinoma cells at or below normal cells indicates the adenocarcinoma is a BAC.

20. The method of claim 19, wherein expression of the gene in the malignant cells is determined by determining copy number of the gene, wherein a copy number higher than 2 indicates increased expression of the gene and a copy number of 2 or less indicates expression of the gene at or below normal cells.

21. A method of deciding a course of treatment for a patient with a cancer, the method comprising
obtaining malignant cells of the cancer from a sample of tissue comprising the cancer, and
comparing expression of a gene in chromosome region 7q21, 7q22, 7q31 or 7q36 in the malignant cells of the cancer with expression of the same gene in normal human cells,
wherein increased expression of the gene in the malignant cells over normal cells indicates the patient should undergo an aggressive course of treatment, and expression of the gene in the malignant cells at or below normal cells indicates the patient should not undergo an aggressive course of treatment.

22. The method of claim 21, wherein expression of the gene in the malignant cells is determined by determining copy number of the gene, wherein a copy number higher than 2 indicates increased expression of the gene and a copy number of 2 or less indicates expression of the gene at or below normal cells.
